(19) United States
(12) Patent Application Publication
(10) Pub. No.: US 2009/0247783 A1
(43) Pub. Date: Oct. 1, 2009

(54) CARBONYLATION PROCESS

(75) Inventors: Joseph Robert Zoeller, Kingsport, TN (US); Mary Kathleen Moore, Jonesborough, TN (US)

Correspondence Address: ERIC D. MIDDLEMAS;EASTMAN CHEMICAL COMPANY, P. O. BOX 511, KINGSPORT, TN, 37662-5075, US

(73) Assignee: EASTMAN CHEMICAL COMPANY, Kingsport, TN (US)

(21) Appl. No.: 12/060,741

(22) Filed: Apr. 1, 2008

(51) Int. Cl. C07C 51/12; C07C 67/37

(52) U.S. Cl. 560/232; 562/519

(57) ABSTRACT

Disclosed is an improved carbonylation process for the production of carboxylic acids, carboxylic acid esters, and/or carboxylic acid anhydrides wherein a carbonylation feedstock compound selected from one or more organic oxygenates such as alcohols, ethers, and esters is contacted with carbon monoxide in the presence of a carbonylation catalyst and one or more onium compounds. The carbonylation process differs from known carbonylation processes in that a halide compound, other than the onium salt, such as a hydrogen halide (typically, hydrogen iodide) and/or an alkyl halide (typically, methyl iodide), extraneous or exogenous to the carbonylation process is not fed or supplied to the process. The process can be improved by using a bidentate ligand comprising two functional groups selected from tertiary amines and tertiary phosphines, such as 2,2′-bipyridine and diphosphine derivatives.

### FIELD OF THE INVENTION

This invention generally pertains to a carbonylation process for producing carboxylic acids, carboxylic acid esters, and/or carboxylic acid anhydrides by contacting a carbonylation feedstock compound selected from one or more organic oxygenates such as alcohols, ethers, and esters with carbon monoxide in the presence of a carbonylation catalyst and one or more onium compounds.

More specifically, this invention pertains to a carbonylation process wherein a halide compound, other than an onium salt compound, such as a hydrogen halide (typically, hydrogen iodide) and/or an alkyl halide (typically, methyl iodide), exogenous or extraneous to the carbonylation process is not fed or supplied to the process. The present carbonylation process thus avoids the handling and storage of hazardous and corrosive hydrogen and alkyl halides.

This invention further pertains to an improved carbonylation process that involves using a bidentate ligand comprising two functional groups selected from tertiary amines and tertiary phosphines to significantly improve the carbonylation rate of oxygenates such as methanol, methyl acetate, and dimethyl ether.

### BACKGROUND OF THE INVENTION

Processes for the manufacture of acetic acid from methanol by carbonylation are operated extensively throughout the world. A thorough review of these commercial processes and other methods for the production of acetyl compounds from single carbon sources are described by Howard et al. in Catalysis Today, 18 (1993) 325-354. All commercial carbonylation processes for the preparation of acetic acid involve feeding methanol, a halogen compound, typically hydrogen iodide and/or methyl iodide, and a solvent such as acetic acid to a reaction zone and contacting the feed materials with carbon monoxide and a Group VIII catalyst, typically a rhodium or iridium catalyst. The liquid reaction mixture is removed from the reaction zone and the product acetic acid and/or other acetyl compound is recovered from the liquid.

In the most important carbonylation processes, i.e., the conversion of methanol to acetic acid and the conversion of methyl acetate to acetic anhydride, hydrogen iodide and/or methyl iodide normally are fed to the reaction zone where the carbonylation reaction occurs. The feed of hydrogen iodide and/or methyl iodide is problematic since the hydrogen iodide and/or methyl iodide are corrosive, must be removed from the product and recycled in subsequent distillation steps, and due to its toxicity and volatility, requires very rigorous and expensive process controls. Elimination of the requirement to add this large volume of methyl iodide would significantly reduce the costs associated with separation and the expensive control equipment associated with safely handling such a volatile and toxic component.

A review of these processes is available in Howard, et. al., Catalysis Today, 18, 325-354 (1993). Included in the Howard et. al. article is a listing of attempts to develop an alkyl halide-free carbonylation system (see pages 345-347). However, all previous attempts have failed to provide a commercially-viable process since alkyl halide-free carbonylation processes give very slow reaction rates, proceeding at about 1% or less of the rates of the commercial process.

### SUMMARY OF THE INVENTION

We have surprisingly developed a carbonylation process that neither utilizes nor requires the introduction or feed of a halide compound, other than an onium salt compound, e.g., hydrogen iodide or an alkyl iodide, in the production of carboxylic acids or esters or anhydrides thereof. We have also surprisingly discovered that the carbonylation rate of such a process can be significantly improved by using a bidentate ligand comprising two functional groups selected from tertiary amines and tertiary phosphines.

The present invention is directed to an improved process for producing a carboxylic acid, a carboxylic acid ester, a carboxylic acid anhydride, or a mixture thereof.

In one embodiment, the process comprises contacting:

(i) a carbonylation feedstock compound selected from alkanols, dialkyl ethers, carboxylic acid esters, and mixtures thereof;

(ii) a Group VIII metal carbonylation catalyst;

(iii) an onium salt compound;

(iv) a bidentate ligand comprising two functional groups selected from tertiary amines and tertiary phosphines; and

(v) carbon monoxide,

in a reaction zone at conditions effective to produce a carbonylation product selected from a carboxylic acid, a carboxylic acid ester, a carboxylic acid anhydride, and a mixture thereof,

wherein a halide compound, other than the onium salt compound, exogenous or extraneous to the process is not added or supplied to the reaction zone.

In a second embodiment, the process comprises:

(a) feeding to a reaction zone
(i) a carbonylation feedstock compound selected from alkanols, dialkyl ethers, carboxylic acid esters, and mixtures thereof,
(ii) a Group VIII metal carbonylation catalyst,
(iii) an onium salt compound,
(iv) a bidentate ligand selected from 2,2′-dipyridine, a 2,2′-dipyridine, a diimine, and a diphosphine, and
(v) optionally, an inert solvent, to provide a reaction zone liquid;

(b) feeding carbon monoxide to the reaction zone liquid under carbonylation conditions of pressure and temperature; and

(c) removing from the reaction zone a crude liquid product comprising a carbonylation product, the carbonylation feedstock compound, the Group VIII metal carbonylation catalyst, the onium salt compound, the bidentate ligand, and the optional inert solvent;

wherein a halide compound, other than the onium salt compound, exogenous or extraneous to the process is not added to the reaction zone.

In a third embodiment, the process comprises:

(a) feeding a carbonylation feedstock compound selected from alkanols, dialkyl ethers, carboxylic acid esters, and mixtures thereof and carbon monoxide to a reaction zone containing a solution comprising a Group VIII metal carbonylation catalyst, an onium salt compound, and a bidentate ligand comprising two functional groups selected from tertiary amines and tertiary phosphines to provide a reaction zone liquid maintained under carbonylation conditions of pressure and temperature; and

(b) removing from the reaction zone a crude gaseous product comprising a carbonylation product, the carbonylation feedstock compound, and carbon monoxide,

wherein a halide compound, other than the onium salt compound, exogenous or extraneous to the process is not added to the reaction zone.

In a fourth embodiment, the process comprises:

(a) feeding a gaseous carbonylation feedstock compound selected from alkanols, dialkyl ethers, carboxylic acid esters, and mixtures thereof and carbon monoxide to a reaction zone containing a Group VIII metal carbonylation catalyst, an onium salt compound, and a bidentate ligand comprising two functional groups selected from tertiary amines and tertiary phosphines (1) deposited on a catalyst support material or (2) in the form of a polymeric material containing quaternary nitrogen groups; and

(b) removing from the reaction zone a crude gaseous product comprising a carbonylation product, the carbonylation feedstock compound, and carbon monoxide,

wherein a halide compound, other than the onium salt compound, exogenous or extraneous to the carbonylation process is not added to the reaction zone.

### DETAILED DESCRIPTION OF THE INVENTION

The carbonylation process according to the present invention generally comprises the step of contacting:

(i) a carbonylation feedstock compound selected from alkanols, dialkyl ethers, carboxylic acid esters, and mixtures thereof;

(ii) a Group VII metal carbonylation catalyst;

(iii) an onium salt compound;

(iv) a bidentate ligand comprising two functional groups selected from tertiary amines and tertiary phosphines; and

(v) carbon monoxide,

in a reaction zone at conditions effective to produce a carbonylation product selected from a carboxylic acid, a carboxylic acid ester, a carboxylic acid anhydride, and a mixture thereof,

wherein a halide compound, other than the onium salt compound, exogenous or extraneous to the process is not added or supplied to the reaction zone.

The carbonylation feedstock compound that may be used in the process of the present invention is selected from alkanols, dialkyl ethers, and alkyl esters of carboxylic acids. The alkanols include substituted alkanols and may contain from 1 to about 10 carbon atoms. Primary alkanols are preferred with methanol being especially preferred. The dialkyl ethers and alkyl carboxylate esters may contain a total of 2 to about 20 carbons. Dimethyl ether and methyl acetate are the most preferred ethers and esters. Depending on the mode of operation of the process of the present invention, the carbonylation feedstock compound may constitute about 5 to 95 weight percent of the reaction medium or solution, i.e., the total weight of the contents of the reaction zone wherein a carbonylation feedstock compound is contacted with carbon monoxide in the presence of a Group VIII metal carbonylation catalyst, an onium salt compound, and a bidentate ligand.

Although the presence of water in the carbonylation feedstock compound is not essential when the feedstock compound is an alkanol, the presence of some water is desirable to suppress formation of carboxylic acid esters and/or dialkyl ethers. When using an alkanol to produce a carboxylic acid, the molar ratio of water to alkanol may be about 0:1 to 10:1, but preferably is in the range of about 0.01:1 to 1:1. When the carbonylation feedstock compound is a carboxylic acid ester or dialkyl ether, the amount of water fed typically is increased to account for the mole of water required for hydrolysis of the alkanol alternative. Therefore, when using either a carboxylic acid ester or dialkyl ether, the mole ratio of water to ester or ether is in the range of about 1:1 to 10:1, but preferably in the range of about 1:1 to 3:1. In the preparation of a carboxylic acid, it is apparent that combinations of alkanol, alkyl carboxylic acid ester, and/or dialkyl ether are equivalent, provided the appropriate amount of water is added to hydrolyze the ether or ester to provide the methanol reactant. When the process is operated to produce a carboxylic acid ester, preferably no water should be added, and a dialkyl ether becomes the preferred feedstock. Further, when an alkanol is used as the feedstock in the preparation of a carboxylic acid ester, it is preferable to remove water.

Products that may be obtained from the present process include carboxylic acids of 2-13 carbons, carboxylic acid anhydrides containing 4 to about 21 carbons, and alkyl carboxylate esters containing 3 to about 21 carbons. The most useful application of the process of the present invention is in production of C₂ to C₄ carboxylic acids such as acetic acid from methanol and propionic acid from ethanol.

The Group VIII metal carbonylation catalyst may be selected from a variety of compounds of the metals in Groups 8, 9, and 10, e.g., Fe, Ru, Os, Co, Rh, Ir, Ni, Pd and Pt, of the Periodic Table of Elements traditionally referred to as the Group VIII metals in prior terminology. Co, Rh, Ir, Ni, and Pd and compounds and complexes thereof are preferred with compounds and complexes of Rh and Ir being especially preferred. Any form of these metals may be used, and they may be used as single components or in combination with one another. The Group VIII metal carbonylation catalysts may be employed in combination with promoters or co-catalysts such as alkali metal compounds, Group 6 metal (Cr, Mo, W) compounds, alkaline earth metal compounds and compounds of zinc, tin, and Lanthanide metals. The Group VIII metal carbonylation catalysts typically are used in concentrations between about 0.0001 mol to 1 mol per kg of reaction medium or solution. The more active of the Group VIII metal carbonylation catalysts typically are used in concentrations of about 0.001 to 0.1 mol per kg of reaction medium or solution.

The Group VIII metal carbonylation catalyst, onium salt, and bidentate ligand may be deposited on a catalyst support material such as carbon or an inorganic oxide such as alumina or silica according to known procedures.

The carbonylation process of the present invention is carried out in the presence of an onium salt comprising a cation selected from quaternary atoms or radicals such as quaternary ammonium, quaternary phosphonium, trialkyl sulfonium, and alkylated sulfoxide. The onium salt compound may be functional and includes protonated forms of the atoms or radicals, especially protonated forms of various tertiary amines and tertiary phosphines. The onium salt may contain any number of carbon atoms, e.g., up to about 60 carbon atoms, and also may contain one or more heteroatoms. The tri- and tetra-alkyl quaternary ammonium and phosphonium salts typically contain a total of about 5 to 40 carbon atoms.

Examples of quaternary ammonium and phosphonium salts include salts of cations having the formula

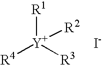

wherein R¹, R², R³, and R⁴ are independently selected from alkyl or substituted alkyl moieties having up to about 20 carbon atoms, cycloalkyl or substituted cycloalkyl having about 5 to about 20 carbon atoms, or aryl or substituted aryl having about 6 to about 20 carbon atoms; and Y is N or P.

The quaternary ammonium salts may also be selected from salts of aromatic, heterocyclic onium cations having the formulas

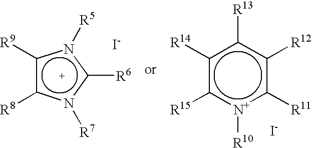

wherein at least one ring atom is a quaternary nitrogen atom and R⁶, R⁸, R⁹, R¹¹, R¹², R¹³, R¹⁴, and R¹⁵ are independently selected from hydrogen, alkyl or substituted alkyl moieties having up to about 20 carbon atoms, cycloalkyl or substituted cycloalkyl having about 5 to about 20 carbon atoms, or aryl or substituted aryl having about 6 to about 20 carbon atoms; and R⁵, R⁷, and R¹⁰ are independently selected from alkyl or substituted alkyl moieties having up to about 20 carbon atoms, cycloalkyl or substituted cycloalkyl having about 5 to about 20 carbon atoms, or aryl or substituted aryl having about 6 to about 20 carbon atoms.

Examples of specific ammonium salts include tetrapentylammonium iodide, tetrahexylammonium iodide, tetraoctylammonium iodide, tetradecylammonium iodide, tetradodecylammonium iodide, tetrapropylammonium iodide, tetrabutylammonium iodide, methyltrioctylammonium iodide, methyltributylammonium iodide, N-octyl-quinuclidinium iodide, N,N′-dimethyl-N,N′-dihexadecylpiperazinium diiodide, dimethyl-hexadecyl-[3-pyrrolidinylpropyl]ammonium iodide, N,N,N,N′,N′,N′-hexa(dodecyl)octane-1,8-diammonium diiodide, N,N,N,N′,N′,N′-hexa(dodecyl)butane-1,4-diammonium diiodide; imidazolium iodides such as 1-butyl-3-methylimidazolium iodide, 1,3-dimethylimidazolium iodide, 1,3,4-trimethylimidazolium iodide, 1,2,3,4,5-penta-methylimidazolium iodide; pyridinium iodides such as N-octylpyridinium iodide, N-methylpyridinium iodide, N-methyl-2-picolinium iodide, N-methyl-3-picolinium iodide, N-methyl-4-picolinium iodide, N-methyl-5-ethyl-2-methyl-pyridinium iodide, N-methyl-3,4-lutidinium iodide; N-methyl quinolinium iodide, N-methyl isoquinolinium iodide or mixtures thereof. Preferred quaternary ammonium iodides include 1-butyl-3-methylimidizolium iodide, N-methylpyridinium iodide, N-methyl-2-methylpyridinium iodide, N-methyl-3-methylpyridinium iodide, N-methyl-4-methylpyridinium iodide, N-methyl-5-ethyl-2-methyl-pyridinium iodide, and 1,3-dimethylimidazolium iodide.

Exemplary phosphonium compounds include tetraoctylphosphonium iodide, tetrabutylphosphonium iodide, triphenyl(hexyl)phosphonium iodide, triphenyl(octyl)phosphonium iodide, tribenzyl(octyl)phosphonium iodide, tribenzyl(dodecyl)phosphonium iodide, triphenyl(decyl)phosphonium iodide, triphenyl(dodecyl)phosphonium iodide, tetrakis(2-methylpropyl)phosphonium iodide, tris(2-methylpropyl)(butyl)phosphonium iodide, triphenyl(3,3-dimethylbutyl)phosphonium iodide, triphenyl(3-methylbutyl)phosphonium iodide, tris(2-methylbutyl)(3-methylbutyl)-phosphonium iodide, triphenyl[2-trimethylsilylethyl]phosphonium iodide, tris(p—chlorophenyl)(dodecyl)phosphonium iodide, hexyltris(2,4,6-trimethylphenyl)phos-phonium iodide, tetradecyltris(2,4,6-trimethylphenyl)phosphonium iodide, dodecyltris(2,4,6-trimethylphenyl)phosphonium iodide, methyltriocytiphosphonium iodide, methyltributylphosphonium iodide, methyltricyclohexylphosphonium iodide, and the like. Preferred phosphonium iodides include methyltriphenylphosphonium iodide, methyltributylphosphonium iodide, methyltriocytiphosphonium iodide, and butyltridodecylphosphonium iodide.

The onium salt may be generated from polymers containing a quaternary or quaternizable phosphine or amine. The onium salt polymer may be derived in whole or part from (or containing polymerized residues of) 2- or 4-vinyl-N-alkylpyridinium halides or 4-(trialkylammonium)styrene halides. For example, a variety of 4-vinyl pyridine polymers and copolymers are available, and may be quaternized or protonated with alky halides or hydrogen halides to generate heterogeneous onium salts. Further, polymers of N-methyl-4-vinylpyridium chloride are commercially available and may be used as—is or are preferably exchanged with iodide by well known means to form the iodide salt. The heterogeneous onium compound may comprise (1) an onium salt compound deposited on a catalyst support material or (2) of a polymeric material containing quaternary nitrogen groups. Examples of such polymeric onium compounds include polymers and co-polymers of vinyl monomers which contain quaternary nitrogen (ammonium) groups. Polymers and copolymers derived from 2- and 4-vinyl-N-alkylpyridinium halides, e.g., poly(4-vinyl-N-methylpyridinium iodide), are specific examples of such polymeric onium salt compounds.

The most preferred onium salts comprise N-alkylpyridinium halides and N,N′- (or 1,3-)dialkylimidazolium halides wherein the alkyl groups contain 1 to about 4 carbon atoms. The onium salts may contain one or more quaternary cations and/or one or more anions. The anion(s) of the onium salts may be selected from a wide variety of species such as halides, carboxylates, tetrafluoroborate, hexahalophosphates, bis (trifluoro-methanesulfonyl)amide [(CF₃SO₂)₂N—], and anionic metal complexes such as (CO)₄Co—, trihalozincates, (ZnX₃—, X═F, Cl, Br, or I), trichlorostannates (SnCl₃-) diododicarbonylrhodate (I) and diiododicarbonyliridate (I) and may be mixtures of anions. However, the most useful anions are the halides and carboxylates or mixtures thereof. Iodide anions are especially preferred. The onium salt typically constitutes about 5 to 95 weight percent of the reaction medium or solution depending on the particular onium salt employed and the mode of operation of the carbonylation process.

The onium salts may be prepared according to various procedures known in the art. The most efficient method for preparing the preferred halide salts is to simply alkylate or protonate the amine or phosphine precursor with an alkyl or hydrogen halide. Due to their ease of preparation and availability of the amine and phosphine precursors, the most preferred onium salts for a liquid-phase operation are selected from the group consisting of quaternary ammonium and phosphonium halides, with the most preferred being halide salts derived from pyridine and imidazole derivatives. The following example illustrates one technique for the preparation of the preferred onium salt-1,3-dimethylimidazolium iodide: To a single neck, 2-liter flask equipped with magnetic-stir bar, nitrogen inlet, condenser and an addition flask, was added 140 grams of 1-methlyimidazole (1.705 moles) and 600 ml of ethyl acetate. Iodomethane (266 grams, 1.876 moles) was added drop-wise over a period of 1 hour to control the exotherm. The reaction mixture was stirred overnight at room temperature. The liquid was decanted and the solids were washed with ethyl acetate and dried on a rotary evaporator for 1 hour at 60° C. under 0.1 mbar of pressure. The 1,3-dimethylimidazolium iodide product (381 g, 1.701 moles, 99.7% mass yield) was a crystalline solid and was spectroscopically pure by NMR. Similar results can be obtained using tetrahydrofuran (THF) as solvent.

The carbonylation process of the present invention is also carried out in the presence of a bidentate ligand comprising two functional groups selected from tertiary amines and tertiary phosphines. The bidentate ligand can have two tertiary amine groups, two tertiary phosphine groups, or one tertiary amine group and one tertiary phosphine group. Preferred bidentate ligands include diphosphines, 2,2′-bipyridines such as 2,2′-bipyridine itself, and diimines.

The diphosphines include those of the general structure:

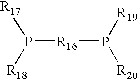

where R₁₆ is a bridging group, normally an alkyl chain of 1 to 6 methylene carbons, but may also be an aryl, biaryl, cycloalkyl, or a heteroatom, such as nitrogen, oxygen, or sulfur, and R₁₇, R₁₈, R₁₉, and R₂₀ are typically aryl, alkyl, cycloalkyl, alkoxy, or phenoxy group containing 1-20 carbons.

Without representing an exhaustive list, specific examples of diphosphines include 1,2-bis-diphenylphosphinoethane; 1,3-bis-diphenylphosphinopropane; 1,4-bis-diphenylphosphinobutane; 1,5-bis-diphenylphosphinopentane; 1,6-bis-diphenylphosphinohexane; 1,2-bis-dicyclohexylphosphinoethane; 1,3-bis-dicyclohexylphosphinopropane; 1,4-bis-dicyclohexylphosphinobutane; 1,2-bis-dimethylphosphinoethane; 1,3-bis-dimethylphosphinopropane; 1,4-bis-dimethylphosphinobutane; 1,2-bis-diisopropylphosphinoethane; 1,3-bis-diisopropylphosphinopropane; 1,4-bis-diisopropylphosphinobutane; 1,2-bis-di-tert-butyl phosphinoethane; 1,3-bis-di-tert-butyl phosphinopropane; 1,4-bis-di-tert-butyl phosphinobutane; 1,2-bis-diphenylphosphinobenzene; N,N-bis-(diphenylphosphino)-amine; 2,2′-diphenylphosphinobiphenyl; and 2,2′-bis-(diphenylphosphino)methyl biphenyl.

The 2,2′-bipyridines include those of the general structure:

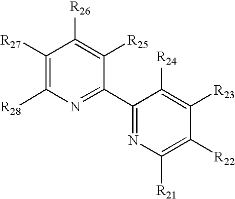

In the most preferred embodiment, R₂₁, R₂₂, R₂₃, R₂₄, R₂₅, R₂₆, R₂₇, and R₂₈ are all hydrogen. However, other useful examples of 2,2′-bipyridines include those where one or more of R₂₁, R₂₂, R₂₃, R₂₄, R₂₅, R₂₆, R₂₇, and R₂₈ are aryl or alkyl containing up to 20 carbon atoms, or a functional group, such as a carboxy, carboalkoxy, hydroxy, alkoxy, aryloxy, and amine. Further, R₂₄ and R₂₅ may be a bridging group, such as an olefin, alkyl, or heteroatom bridge.

Specific examples of 2,2′-bypiridines include 2,2′-bipyridine; 1,10-phenanthroline; 3,3′-dimethyl-2,2′-bipyridine; 4,4′-dimethyl-2,2′-bipyridine; 5,5′-dimethyl-2,2′-bipyridine; and 4,7-diphenyl-1,10-phenanthroline.

The diimines include those of the general structure:

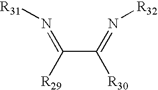

where R₂₉, R₃₀, R₃₁, and R₃₂ are normally alkyl, cycloalkyl, or aryl groups of up to 20 carbons. They may also be functional groups such as a carboxy, carboalkoxy, hydroxy, alkoxy, aryloxy, and amine. Further, R₂₉ and R₃₀ may be bridging groups, including particularly alkyl bridges.

Specific examples of diimines include 2,3-bis-(2,6-di-isopropyl-phenylimino)-butane; 2,3-bis-mesitylimino-butane; 2,3-bis-phenylimino butane; 1,2-bis-(2,6-di-isopropyl-phenylimino)-cyclohexane; 3,4-bis-(2,6-di-isopropyl-phenylimino)-hexane; 1,2-diphenyl-1,2-bis-(2,6-di-isopropyl-phenylimino)-ethane; 2,3-bis-cyclohexylimino-butane; 1,2-diphenyl-1,2-bis-cyclohexylmino ethane; 1,2-diphenyl-1,2-bis-cyclohexylmino ethane; dimethylglyoxime; and diphenylglyoxime.

The molar ratio of the bidentate ligand to the Group VIII metal catalyst may range from 0.1:1 to 50:1, but is preferably 1:1 to 5:1, with 1:1 to 2:1 being most preferred.

The carbon monoxide may be fed to the reaction or carbonylation zone either as purified carbon monoxide or as carbon monoxide including other gases. The carbon monoxide need not be of high purity and may contain from about 1% by volume to about 100% by volume carbon monoxide, and preferably from about 70% by volume to about 99% by volume carbon monoxide. The remainder of the gas mixture may include such gases as nitrogen, hydrogen, water, and parafinic hydrocarbons having from one to four carbon atoms. Although hydrogen is not part of the reaction stoichiometry, hydrogen may be useful in maintaining optimal catalyst activity. Therefore, the preferred molar ratio of carbon monoxide to hydrogen is in the range of about 99:1 to about 2:1, but ranges with even higher hydrogen levels are also useful. The amount of carbon monoxide useful for the carbonylation reaction ranges from a molar ratio of about 0.1:1 to about 1,000:1 of carbon monoxide to alcohol, ether, or ester equivalents with a more preferred range being from about 0.5:1 to about 100:1, and a most preferred range from about 1.0:1 to about 20:1.

The carbonylation conditions of pressure and temperature may vary significantly depending upon various factors such as, for example, the mode of operation, the Group VIII metal catalyst employed, the process apparatus utilized, and the degree of conversion of the carbonylation feedstock that is desired. For example, the process may be operated under a pressure (total) ranging from atmospheric pressure to 250 bar gauge (barg; 3700 pounds per square inch gauge—psig). However, pressures (total) in the range of about 5 to 100 barg (72.5 to 1450 psig) are more typical with pressures in the range of about 10 to 80 barg being preferred when using the preferred rhodium as the Group VIII metal carbonylation catalyst. The process temperature may range from about 50 to 300° C., although temperatures in the range of about 150 to 250° C. are more typical.

In the carbonylation process provided by the present invention, a halide compound, other than the onium salt compound, such as hydrogen halide or an alkyl halide, exogenous or extraneous to the carbonylation process is not added or supplied to the reaction zone. For example, fresh hydrogen halide and/or fresh alkyl halide are not fed to the reaction zone of the process. Minor amounts, i.e., minor as compared to known processes, of such halides, e.g., methyl iodide, may form during operation of the process by reaction of a feedstock compound, or fragment of a feedstock compound, with a halide anion of the onium salt compound.

In continuous operation of the carbonylation process, a low-boiling stream can be recovered from the product recovery and refining section of the process. This low boiling stream can be recycled to the reaction zone of the carbonylation process.

The carbonylation process provided by the present invention provides a means for preparing a carbonylation product selected from carboxylic acids, carboxylic acid esters, carboxylic acid anhydrides, or a mixture of any two or more thereof. The process may be carried out using any of a variety of operational modes.

For example, in mode (1), the process comprises:

(a) feeding to a reaction zone
(i) a carbonylation feedstock compound selected from alkanols, dialkyl ethers, carboxylic acid esters, and mixtures thereof,
(ii) a Group VIII metal carbonylation catalyst,
(iii) an onium salt compound,
(iv) a bidentate ligand selected from 2,2′-dipyridine, a 2,2′-dipyridine, a diimine, and a diphosphine, and
(v) optionally, an inert solvent, to provide a reaction zone liquid;

(b) feeding carbon monoxide to the reaction zone liquid under carbonylation conditions of pressure and temperature; and

(c) removing from the reaction zone a crude liquid product comprising a carbonylation product, the carbonylation feedstock compound, the Group VIII metal carbonylation catalyst, the onium salt compound, the bidentate ligand, and the optional inert solvent;

wherein a halide compound, other than the onium salt compound, exogenous or extraneous to the process is not added to the reaction zone.

Mode (1) can be run at a temperature of about 100 to 250° C. and a pressure (total) of about 5 to 80 barg. The reaction zone liquid typically comprises about 10 to 80 weight percent of the carbonylation feedstock compound, about 10 to 80 weight percent of the carbonylation product, about 10 to 80 weight percent of the onium salt, about 0.002 to 0.2 weight percent (20-2,000 ppm) of the catalyst metal, about 0.002 to 1 weight percent (20-10,000 ppm) of the bidentate ligand, and about 0 to 50 weight percent of the optional inert solvent. The optional inert solvent is preferably a carboxylic acid. Preferably, the carboxylic acid corresponds to the carbonylation product, e.g., acetic acid, when the carbonylation product is acetic acid or acetic anhydride.

The carbonylation product can be recovered from the crude liquid product removed from the reaction zone by known techniques. The remainder of the crude product would comprise a low-boiling fraction comprising unreacted carbonylation feedstock compound and a high-boiling fraction comprising the Group VIII metal carbonylation catalyst, the onium salt compound, the bidentate ligand, and the optional inert solvent. Normally, some or all of the low-boiling and high-boiling fractions are recovered from the crude liquid product and recycled directly or indirectly to the reaction zone. Thus, the continuous operation of mode (1) of the process of the present invention can include the steps of:
(iii) refining the crude liquid carbonylation product to recover (1) the carbonylation product, (2) a low-boiling fraction comprising the carbonylation feedstock compound and (3) a high-boiling fraction comprising the Group VIII metal carbonylation catalyst, the onium salt compound, the bidentate ligand, and the optional inert solvent; and
(iv) recycling the low-boiling and high-boiling fractions to the reaction zone.

In mode (2), the process comprises:

(a) feeding a carbonylation feedstock compound selected from alkanols, dialkyl ethers, carboxylic acid esters, and mixtures thereof and carbon monoxide to a reaction zone containing a solution comprising a Group VIII metal carbonylation catalyst, an onium salt compound, and a bidentate ligand comprising two functional groups selected from tertiary amines and tertiary phosphines to provide a reaction zone liquid maintained under carbonylation conditions of pressure and temperature; and

(b) removing from the reaction zone a crude gaseous product comprising a carbonylation product, the carbonylation feedstock compound, and carbon monoxide,

wherein a halide compound, other than the onium salt compound, exogenous or extraneous to the process is not added to the reaction zone.

In mode (2), the process typically operates at a temperature range of 100 to 250° C. Other examples of operable temperature ranges include 120 to 240° C. and 150 to 240° C. The pressure (total) of the reaction zone typically is maintained in the range of about 1 to 50 barg. The reaction zone liquid may comprise a solution of the Group VIII metal compound in a melt of the onium salt compound, or it may comprise a solution of the Group VIII metal compound and the onium salt compound in a high-boiling, i.e., substantially non-volatile under reaction conditions, solvent. Examples of such high-boiling solvents include sulfoxides and sulfones, e.g., dimethyl sulfoxide and sulfolane; amides, e.g., N-methyl-2-pyrrolidinone (NMP), dimethylacetamide, C₆ to C₃₀ carboxylic acids; aromatic hydrocarbons, e.g., 2-methylnaphthalene; and high-boiling, saturated hydrocarbons, e.g., decalin, dodecane.

While the mode (2) reaction nominally is a vapor phase process, the liquid reaction medium or reaction zone typically contains at least a portion of the carbonylation feedstock and product as a solution. Typically, the reaction medium comprises about 1 to 40 weight percent of the carbonylation feedstock compound, about 1 to 60 weight percent of the carbonylation product, about 10 to 100 weight percent of the onium salt, about 0.002 to 0.2 weight percent (20-2,000 ppm) of the catalyst metal, about 0.002 to 1 weight percent (20-10,000 ppm) of the bidentate ligand, and 0 to about 50 weight percent the high-boiling solvent. The carbonylation feedstock compound may be fed to the mode (2) process either as a vapor or liquid. A liquid feed is converted to a vapor within the reaction zone or preferably in a preheated section of the process apparatus. The effluent from the mode (2) process is a vapor typically comprised of carbonylation product, unconverted carbonylation feedstock compound, and carbon monoxide.

US 2007/0293695 A1 describes operation of a process similar to that of mode (2) of the present invention.

Any onium salt, catalyst, bidentate ligand, optional inert solvent, carbonylation feedstock, or low-boiling components or intermediates present in the gaseous product removed from the reaction zone may be separated during product recovery/purification and returned to the reaction zone. Thus, the continuous operation of mode (2) can include the steps of:
(iii) refining the crude gaseous carbonylation product to recover (1) the carbonylation product and (2) a low-boiling fraction comprising the carbonylation feedstock compound; and
(iv) recycling the low-boiling fraction to the reaction zone.

In mode (3), the process comprises:

(a) feeding a gaseous carbonylation feedstock compound selected from alkanols, dialkyl ethers, carboxylic acid esters, and mixtures thereof and carbon monoxide to a reaction zone containing a Group VIII metal carbonylation catalyst, an onium salt compound, and a bidentate ligand comprising two functional groups selected from tertiary amines and tertiary phosphines (1) deposited on a catalyst support material or (2) in the form of a polymeric material containing quaternary nitrogen groups; and

(b) removing from the reaction zone a crude gaseous product comprising a carbonylation product, the carbonylation feedstock compound, and carbon monoxide,

wherein a halide compound, other than the onium salt compound, exogenous or extraneous to the carbonylation process is not added to the reaction zone.

Mode (3) can be operated similarly to mode (2), except that both the Group VIII metal carbonylation catalyst, the bidentate ligand, and the onium compound are in solid form.

U.S. Pat. No. 6,452,043-B1 and US-2005/0049434-A1 describe a vapor phase operation, albeit with an added alkyl halide.

Any onium salt, catalyst, bidentate ligand, optional inert solvent, carbonylation feedstock, or low-boiling components or intermediates entrained in the vapor effluent product can be separated during purification and returned to the reaction zone. Thus, the continuous operation of mode (3) can include the steps of:
(iii) refining the crude gaseous carbonylation product to recover (1) the carbonylation product and (2) a low-boiling fraction comprising the carbonylation feedstock compound; and
(iv) recycling the low-boiling fraction to the reaction zone.

This invention can be further illustrated by the following examples, although it will be understood that these examples are included merely for purposes of illustration and are not intended to limit the scope of the invention.

### EXAMPLES

All percentages below are by weight except for the 5% hydrogen in carbon monoxide, which is by volume. The experiments described in the following examples were carried out in an autoclave constructed of Hastelloy® C-276 alloy. GC analysis was conducted by dissolving a weighed portion of the product in propionic acid with decane added as an internal standard to provide the weight percent of each component.

### Example 1

To a 300 mL autoclave was added 0.396 g (1.5 millimole—mmol) of RhCl₃.3H₂O, 112.0 g (0.507 mol) of N-methylpyridinium iodide, 30.0 g (0.5 mol) of acetic acid, and 64.0 g (2.0 mol) of methanol. The mixture was heated to 190° C. under 17.2 barg (250 psig) of 5% hydrogen in carbon monoxide. Upon reaching 190° C., the gas feed was switched to 100% CO and the pressure adjusted to 51.7 barg (750 psig) using 100% CO. The temperature and pressure were maintained for 5 hours using 100% CO as needed to maintain pressure. After 5 hours, the reaction was cooled, vented, and the product transferred to a sample bottle. GC analysis of the product showed that the mixture contained 0.25% methyl acetate, 0.04% methanol, and 55.84% acetic acid. This represents 2.33 moles of acetic acid representing a net production of acetic acid=1.83 moles after accounting for acetic acid in the original solution and 0.008 mol of methyl acetate along with 0.035 moles of unreacted methanol. No methyl iodide was detected in the product by GC analysis.

### Example 2

To a 300 mL autoclave was added 0.396 g (1.5 mmol) of RhCl₃.3H₂O, 112.0 g (0.507 mol) of N-methylpyridinium iodide, 30.0 g (0.5 mol) of acetic acid, and 64.0 g (2.0 mol) of methanol. The mixture was heated to 190° C. under 17.2 barg (250 psig) of 5% hydrogen in carbon monoxide. Upon reaching 190° C., the pressure was adjusted to 51.7 barg (750 psig) using 5% hydrogen in carbon monoxide. The temperature and pressure were maintained for 5 hours using 5% hydrogen in carbon monoxide as needed to maintain pressure. After 5 hours, the reaction was cooled, vented, and the product transferred to a sample bottle. GC analysis of the product showed that the mixture contained 0.09% methyl acetate, and 57.42% acetic acid. This represents 2.52 moles of acetic acid representing a net production of acetic acid=2.02 moles after accounting for acetic acid in the original solution and 0.003 mol of methyl acetate. Neither methyl iodide nor methanol was detected in the product by GC analysis. This example shows that the conversion and selectivity may be enhanced by the presence of hydrogen.

### Example 3

To a 300 mL autoclave was added 0.396 g (1.5 mmol) of RhCl₃.3H₂O, 112.0 g (0.507 mol) of N-methylpyridinium iodide, 3.0 g of water, 30.0 g (0.5 mol) of acetic acid, and 64.0 g (2.0 mol) of methanol. The mixture was heated to 190° C. under 17.2 barg (250 psig) of 5% hydrogen in carbon monoxide. Upon reaching 190° C., the gas feed was switched to 100% CO and the pressure adjusted to 51.7 barg (750 psig) using 100% CO. The temperature and pressure were maintained for 5 hours using 100% CO as needed to maintain pressure. After 5 hours, the reaction was cooled, vented, and the product transferred to a sample bottle. GC analysis of the product shows that the mixture contained 2.83% methyl acetate, 0.04% methanol, and 57.42% acetic acid. This represents 2.5 moles of acetic acid, or a net production of acetic acid=2.0 moles after accounting for acetic acid in the original reaction zone solution, and 0.103 mol of methyl acetate along with 0.003 moles of unreacted methanol. Only a small amount of methyl iodide (0.15% or 0.003 mol) was detected in the product by GC analysis.

### Example 4

To a 300 mL autoclave was added 0.396 g (1.5 mmol) of RhCl₃.3H₂O, 89.6 g (0.40 mol) of N,N′-dimethylimidazolium iodide, 30.0 g (0.5 mol) of acetic acid, and 64.0 g (2.0 mol) of methanol. The mixture was heated to 190° C. under 17.2 barg (250 psig) of 5% hydrogen in carbon monoxide. Upon reaching 190° C., the gas feed was switched to 100% CO and the pressure adjusted to 51.7 barg (750 psig) using 100% CO. The temperature and pressure were maintained for 5 hours using 100% CO as needed to maintain pressure. After 5 hours, the reaction was cooled, vented, and the product transferred to a sample bottle. GC analysis of the product showed that the mixture contained 0.10% methyl acetate, 0.36% methanol, and 59.16% acetic acid. This represents 2.32 moles of acetic acid, a net production of acetic acid=1.82 moles after accounting for acetic acid in the starting reaction zone solution, and 0.003 mol of methyl acetate along with 0.027 moles of unreacted methanol. Only a trace (0.05%, 0.8 mmol) of methyl iodide was detected in the product by GC analysis.

### Example 5

To a 300 mL autoclave was added 0.396 g (1.5 mmol) of RhCl₃.3H₂O, 89.6 g (0.40 mol) of N,N′-dimethylimidazolium iodide, 30.0 g (0.5 mol) of acetic acid, and 64.0 g (2.0 mol) of methanol. The mixture was heated to 190° C. under 17.2 barg (250 psig) of 5% hydrogen in carbon monoxide. Upon reaching 190° C., the pressure was adjusted to 51.7 barg (750 psig) using 5% hydrogen in carbon monoxide. The temperature and pressure were maintained for 5 hours using 5% hydrogen in carbon monoxide as needed to maintain pressure. After 5 hours, the reaction was cooled, vented, and the product transferred to a sample bottle. GC analysis of the product indicated that the mixture contained 0.24% methyl acetate and 51.85% acetic acid. This represents 1.94 moles of acetic acid, a net production of acetic acid=1.44 moles after accounting for acetic acid in the original solution, and 0.007 mol of methyl acetate. Neither methyl iodide nor methanol was detected in the product by GC analysis.

### Example 6

To a 300 mL autoclave was added 0.396 g (1.5 mmol) of RhCl₃.3H₂O, 89.6 g (0.40 mol) of N,N-dimethylimidazolium iodide, 3.0 g of water, 30.0 g (0.5 mol) of acetic acid, and 64.0 g (2.0 mol) of methanol. The mixture was heated to 190° C. under 17.2 barg (250 psig) of 5% hydrogen in carbon monoxide. Upon reaching 190° C., the gas feed was switched to 100% CO and the pressure adjusted to 61.7 barg (750 psig) using 100% CO. The temperature and pressure were maintained for 5 hours using 100% CO as needed to maintain pressure. After 5 hours, the reaction was cooled, vented, and the product transferred to a sample bottle. GC analysis of the product indicated that the mixture contained 0.21% methyl acetate, 0.76% methanol, and 57.36% acetic acid. This represents 2.26 moles of acetic acid, a net production of acetic acid=1.76 moles after accounting for acetic acid in the initial reaction zone solution, and 0.007 mol of methyl acetate along with 0.057 moles of unreacted methanol. A small amount of methyl iodide (0.38%, 0.003 mol) was detected in the product mixture by GC analysis.

### Example 7

To a 300 mL autoclave was added 0.396 g (1.5 mmol) of RhCl₃.3H₂O, 106.4 g (0.40 mol) of N-butyl-N′-methylimidazolium iodide, 30.0 g (0.5 mol) of acetic acid, and 64.0 g (2.0 mol) of methanol. The mixture was heated to 190° C. under 17.2 barg (250 psig) of 5% hydrogen in carbon monoxide. Upon reaching 190° C., the gas feed was switched to 100% CO and the pressure adjusted to 51.7 barg (750 psig) using 100% CO. The temperature and pressure were maintained for 5 hours using 100% CO as needed to maintain pressure. After 5 hours, the reaction was cooled, vented, and the product transferred to a sample bottle. GC analysis of the product indicated that the mixture contained 57.43% acetic acid. This represents 2.44 moles of acetic acid, a net production of acetic acid=1.94 moles after accounting for acetic acid in the starting reaction zone solution. No methyl iodide, methanol, or methyl acetate was detected in the product mixture by GC analysis.

### Example 8

To a 300 mL autoclave was added 0.396 g (1.5 mmol) of RhCl₃.3H₂O, 131.0 g (0.40 mol) of tributyl(methyl)ammonium iodide, 30.0 g (0.5 mol) of acetic acid, and 64.0 g (2.0 mol) of methanol. The mixture was heated to 190° C. under 17.2 barg (250 psig) of 5% hydrogen in carbon monoxide. Upon reaching 190° C., the gas feed was switched to 100% CO and the pressure adjusted to 51.7 barg (750 psig) using 100% CO. The temperature and pressure were maintained for 5 hours using 100% CO as needed to maintain pressure. After 5 hours, the reaction was cooled, vented, and the product transferred to a sample bottle. GC analysis of the product showed that the mixture contained 0.11% methyl acetate, 0.34% methanol, and 42.69% acetic acid. This represents 1.88 moles of acetic acid, a net production of acetic acid=1.38 moles after accounting for acetic acid in the initial reaction zone solution, and 0.004 mol of methyl acetate. No methyl iodide was detected in the product mixture by GC analysis.

### Example 9

To a 300 mL autoclave was added 0.396 g (1.5 mmol) of RhCl₃.3H₂O, 205.4 g (0.40 mol) of trioctylmethyl phosphonium iodide, 30.0 g (0.5 mol) of acetic acid, and 64.0 g (2.0 mol) of methanol. The mixture was heated to 190° C. under 17.2 barg (250 psig) of 5% hydrogen in carbon monoxide. Upon reaching 190° C., the gas feed was switched to 100% CO and the pressure adjusted to 51.7 barg (750 psig) using 100% CO. The temperature and pressure were maintained for 5 hours using 100% CO as needed to maintain pressure. After 5 hours, the reaction was cooled, vented, and the product transferred to a sample bottle. GC analysis of the product showed that the mixture contained 12.15% methyl acetate, 1.81% methanol, and 11.73% acetic acid. This represents 0.60 moles of acetic acid, a net production of acetic acid=0.1 moles after accounting for acetic acid in the starting reaction zone solution, and 0.51 mol of methyl acetate. This represents a net acetyl(methyl acetate+acetic acid) production of 0.61 mol. A trace (0.19%, 0.004 mol) of methyl iodide was detected in the product mixture by GC analysis.

### Example 10

To a 300 mL autoclave was added 1.39 g (1.5 mmol) of chloro tris-(triphenylphosphine) rhodium, 112.0 g (0.507 mol) of N-methylpyridinium iodide, 30.0 g (0.5 mol) of acetic acid, and 64.0 g (2.0 mol) of methanol. The mixture was heated to 190° C. under 17.2 barg (250 psig) of 5% hydrogen in carbon monoxide. Upon reaching 190° C., the gas feed was switched to 100% CO and the pressure adjusted to 51.7 barg (750 psig) using 100% CO. The temperature and pressure were maintained for 5 hours using 100% CO as needed to maintain pressure. After 5 hours, the reaction was cooled, vented, and the product transferred to a sample bottle. GC analysis of the product showed that the mixture contained 0.63% methyl acetate and 49.64% acetic acid. No methanol was detected. This represents 2.07 moles of acetic acid, a net production of acetic acid=1.57 moles after accounting for acetic acid in the initial reaction zone solution, and 0.02 mol of methyl acetate. Only small amount of methyl iodide (0.06%, 0.001 mol) was detected in the product by GC analysis.

### Example 11

To a 300 mL autoclave was added 1.39 g (1.5 mmol) of chloro tris-(triphenylphosphine) rhodium, 1.18 g (4.5 mmol) of triphenylphosphine, 112.0 g (0.507 mol) of N-methylpyridinium iodide, 30.0 g (0.5 mol) of acetic acid, and 64.0 g (2.0 mol) of methanol. The mixture was heated to 190° C. under 17.2 barg (250 psig) of 5% hydrogen in carbon monoxide. Upon reaching 190° C., the gas feed was switched to 100% CO and the pressure adjusted to 51.7 bar (750 psi) using 100% CO. The temperature and pressure were maintained for 5 hours using 100% CO as needed to maintain pressure. After 4 hours, the reaction was cooled, vented, and the product transferred to a sample bottle. GC analysis of the product showed that the mixture contained 0.35% methyl acetate and 52.19% acetic acid. No methanol was detected. This represents 2.23 moles of acetic acid, a net production of acetic acid=1.73 moles after accounting for acetic acid in the initial reaction zone solution, and 0.02 mol of methyl acetate. Only a small amount of methyl iodide (0.05%, 0.001 mol) was detected in the product by GC analysis.

### Example 12

To a 300 mL autoclave was added 0.396 g (1.5 mmol) of RhCl₃.3H₂O, 117.5 g (0.50 mol) of N-ethylpyridinium iodide, 30.0 g (0.5 mol) of acetic acid, and 64.0 g (2.0 mol) of methanol. The mixture was heated to 190° C. under 17.2 barg (250 psig) of 5% hydrogen in carbon monoxide. Upon reaching 190° C., the gas feed was switched to 100% CO and the pressure adjusted to 51.7 barg (750 psig) using 100% CO. The temperature and pressure were maintained for 5 hours using 100% CO as needed to maintain pressure. After 5 hours, the reaction was cooled, vented, and the product transferred to a sample bottle. GC analysis of the product indicated that the mixture contained 0.44% methyl acetate and 51.22% acetic acid. This represents 2.14 moles of acetic acid, a net production of acetic acid=1.64 moles after accounting for acetic acid in the initial reaction zone solution, and 0.015 mol of methyl acetate. No methyl iodide or unreacted methanol was detected in the product by GC analysis.

### Example 13

A 300 mL autoclave was modified to maintain a temperature control over the entire reactor, and then connected via a U-tube to a high pressure condenser constructed of Hastelloy® C-276 alloy such that the vapors from the autoclave fed to the top of the chilled (20° C.) condenser. To collect the condensate, the end of the condenser was connected to a high pressure receiver constructed of Hastelloy® C-276 alloy which was equipped with a backpressure regulator at the top of the receiver to allow pressure control in the system and a valve at the bottom to allow the receiver to be drained. To the reactor/autoclave was added 2.0 g of solid RhCl₃.3H₂O followed by a solution of 125 g (0.60 mol) of N,N′-dimethylimidazolium iodide in 60 g of acetic acid. The autoclave was sealed, and the system was flushed first with nitrogen and then 5% hydrogen in carbon monoxide. After flushing with 5% hydrogen in carbon monoxide, the feed gas was switched to pure carbon monoxide and fed at a rate of 0.90 mol/hour with the backpressure set to maintain a pressure of 17.2 barg (250 psig) in the reactor. The reactor was heated to 190° C., and upon reaching 190° C., methanol was fed at a rate of 24 ml/hour (0.59 mol/hour, CO/MeOH mole ratio=1.5/1). The condensate was collected periodically and analyzed by GC for a period of 8 days. The daily production rate of products is summarized in Table I below wherein “Temp” is the temperature in ° C. of the autoclave reaction zone solution, “MeI” is the moles of methyl iodide detected in the product solution, “MeOAc” is the moles of methyl acetate present in the product solution, “HOAc” is the moles of acetic acid present in the product solution, and “Total Acetyls” is the total moles of methyl acetate and acetic acid present in the product solution. The 60 grams of acetic acid added as solvent in the initial reaction zone solution was subtracted from the acetic acid present in the crude product obtained after the first day of operation. This example demonstrates the operation of the process in a continuous mode using a vapor takeoff reactor similar to that described in U.S. Pat. No. 6,916,951-B1.

TABLE I

Moles of
Product Produced/Day

| | | | | | Total |
|---|---|---|---|---|---|
| Day | Temp | Mel | MeOAc | HOAc | Acetyl |
| 1 | 190 | 0.0383 | 3.90 | 4.23 | 8.13 |
| 2 | 190 | 0.0053 | 1.49 | 0.57 | 2.06 |
| 3 | 190 | 0.0008 | 2.22 | 0.45 | 2.67 |
| 4 | 190 | 0.0014 | 2.56 | 0.62 | 3.18 |
| 5 | 190 | 0.0016 | 2.53 | 0.62 | 3.15 |
| 6 | 190 | 0.0046 | 2.53 | 0.80 | 3.33 |
| 7 | 205 | 0.0130 | 3.35 | 1.26 | 4.61 |
| 8 | 205 | 0.0038 | 2.27 | 0.86 | 3.13 |
| 8 Day | | 0.0688 | 20.85 | 9.41 | 30.26 |
| Total | | | | | |

### Example 14

A 300 mL autoclave was modified to maintain a temperature control over the entire reactor, and then connected via a U-tube to a high pressure condenser constructed of Hastelloy® C-276 alloy such that the vapors from the autoclave fed to the top of the chilled (20° C.) condenser. To collect the condensate, the end of the condenser was connected to a high pressure receiver constructed of Hastelloy® C-276 alloy which was equipped with a backpressure regulator at the top of the receiver to allow pressure control in the system and a valve at the bottom to allow the receiver to be drained. To the reactor/autoclave was added 2.0 g of solid RhCl₃.3H₂O followed by a solution of 125 g (0.56 mol) of N-methylpyridinium iodide in 60 g of acetic acid. The autoclave was sealed and the system was flushed first with nitrogen and then 5% hydrogen in carbon monoxide. After flushing with 5% hydrogen in carbon monoxide, the feed gas was switched to pure carbon monoxide and fed at a rate of 0.90 mol/hour with the backpressure set to maintain a pressure of 17.2 barg (250 psig) in the reactor. The reactor was heated to 190° C., and upon reaching 190° C., methanol was fed at a rate of 24 ml/hour (0.59 mol/hour, CO/MeOH mole ratio=1.5/1). The condensate was collected periodically and analyzed by GC for a period of 6 days. The daily production rate of products is summarized in Table II below wherein “Temp” is the temperature in ° C. of the autoclave reaction zone solution, “MeI” is the moles of methyl iodide detected ion the product solution, “MeOAc” is the moles of methyl acetate present in the product solution, “HOAc” is the moles of acetic acid present in the product solution, and “Total Acetyls” is the total moles of methyl acetate and acetic acid present in the product solution. The 60 grams of acetic acid added as solvent in the initial reaction zone solution was subtracted from the acetic acid present in the crude product obtained after the first day of operation. This example demonstrates the operation of the process in a continuous mode using a vapor takeoff reactor similar to that described in U.S. Pat. No. 6,916,951-B1.

TABLE II

Moles of Product
Produced/Day

| | | | | | Total |
|---|---|---|---|---|---|
| Day | Temp | Mel | MeOAc | HOAc | Acetyl |
| 1 | 190 | 0.042 | 4.37 | 4.38 | 8.76 |
| 2 | 190 | 0.011 | 4.22 | 2.31 | 6.53 |
| 3 | 190 | 0.014 | 4.32 | 2.13 | 6.45 |
| 4 | 190 | 0.022 | 4.29 | 2.91 | 7.20 |
| 5 | 205 | 0.023 | 3.90 | 4.52 | 8.42 |
| 6 | 205 | 0.026 | 4.10 | 4.45 | 8.55 |
| 6 Day | | 0.138 | 25.21 | 20.70 | 45.91 |
| Total | | | | | |

### Example 15

To a 300 mL autoclave was added 0.396 g (1.5 mmol) of RhCl₃.3H₂O, 112.0 g (0.507 mol) of N-methylpyridinium iodide, 30.0 g (0.5 mol) of acetic acid, and 74.0 g (1.0 mol) of methyl acetate. The mixture was heated to 190° C. under 17.2 barg (250 psig) of 5% hydrogen in carbon monoxide. Upon reaching 190° C., the pressure was adjusted to 51.7 barg (750 psig) using 5% hydrogen in CO. The temperature and pressure were maintained for 5 hours using 5% hydrogen in CO as needed to maintain pressure. After 5 hours, the reaction was cooled, vented, and the product transferred to a sample bottle. GC analysis of the product showed that the mixture contained 1.31% methyl acetate, 19.55% acetic anhydride, 32.40% acetic acid, and 1.70% of ethylidene diacetate (1,1-diacetoxyethane). No methyl iodide was detected in the product by GC analysis. This example demonstrates that the process is applicable to the synthesis of acetic anhydride using methyl acetate as the carbonylation feedstock.

### Example 16

To a 300 mL Hastelloy® C-276 autoclave was added 0.500 g (1.4 mmol) of Ir(CO)₂(acetylacetonate), 88.4 g (0.400 mol) of N-methylpyridinium iodide, 30.0 g (0.5 mol) of acetic acid, and 64.0 g (2.0 mol) of methanol. The mixture was heated to 190° C. under 41.4 barg (600 psig) of 5% hydrogen in carbon monoxide. Upon reaching 190° C., the pressure was adjusted to 51.7 barg (750 psi) using 5% hydrogen in carbon monoxide. The temperature and pressure were maintained for 5 hours using 5% hydrogen in carbon monoxide as needed to maintain pressure. After 5 hours, the reaction was cooled, vented, and the product transferred to a sample bottle. GC analysis of the product indicated that the mixture contained 0.159 wt % methyl acetate and 40.702 wt % acetic acid. This represents 0.685 moles of acetic acid (net production of acetic acid=0.185 moles after accounting for acetic acid in the original solution) and 0.002 mol of methyl acetate. Neither methyl iodide nor methanol was detected in the product by GC analysis.

### Example 17

This example demonstrates a measurement of the rate for the carbonylation reaction using a liquid sampling process. To a 300 mL autoclave equipped with a liquid sampling loop and a high pressure addition funnel was added 0.132 g (0.5 mmol) of RhCl₃.3H₂O, 110.5 g (0.50 mol) of N-methylpyridinium iodide, and 45.0 g (0.75 mol) of acetic acid. To the addition funnel was added 64.0 g (2.0 mol) of methanol. The autoclave was flushed with nitrogen and was then heated to 190° C. under 17.2 barg (250 psig) of 5% hydrogen in carbon monoxide and the addition funnel heated to 150° C. Upon reaching 190° C., the gas feed was switched to 100% CO the methanol was added by pressurizing the addition funnel. Immediately upon completing the liquid addition, the pressure was adjusted to 51.7 barg (750 psig) using 100% CO. Samples were removed from the autoclave every 30 minutes for a period of 5 hrs. The temperature and pressure were maintained using 100% CO as needed to maintain pressure. GC analysis of the liquid products is shown in Table III below. Included in this chart is the number of moles of the acetyl components, methyl acetate, and acetic acid. The net acetyls produced is represented by the total of the acetic acid and methyl acetate present minus the 0.75 mol of acetic acid added at the start of the reaction. The rate of production in this reaction (determined from the slope of a graph of net moles of acetyl produced moles vs. time) was 0.107 mol/h representing a space time yield of 0.49 mol acetyl/L-h and a Rh turnover frequency of 213 mol of acetyl/mol-Rh/h.

TABLE III

| | GC Analysis | | | GC Analysis | | Net Acetyls |
|---|---|---|---|---|---|---|
| Time | (wt %) | | | (moles) | | Produced |
| (hrs) | MeOH | MeOAc | AcOH | MeOAc | AcOH | (moles) |
| 0.5 | 10.8 | 13.7 | 10.2 | 0.41 | 0.37 | 0.03 |
| 1.0 | 8.4 | 19.5 | 8.1 | 0.59 | 0.30 | 0.14 |
| 1.5 | 6.8 | 26.0 | 8.8 | 0.82 | 0.34 | 0.40 |
| 2.0 | 5.2 | 28.3 | 10.2 | 0.90 | 0.40 | 0.55 |
| 2.5 | 3.5 | 28.9 | 12.3 | 0.93 | 0.49 | 0.67 |
| 3.0 | 2.3 | 17.1 | 13.3 | 0.53 | 0.51 | 0.28 |
| 3.5 | 2.0 | 25.1 | 17.1 | 0.82 | 0.69 | 0.76 |
| 4.0 | 1.6 | 18.1 | 18.7 | 0.57 | 0.73 | 0.56 |
| 4.5 | 1.3 | 15.3 | 20.7 | 0.49 | 0.81 | 0.55 |
| 5.0 | 1.1 | 13.9 | 22.5 | 0.44 | 0.89 | 0.58 |

### Example 18

This example demonstrates that the addition of a diamine-based bidentate ligand, specifically 2,2′-bipyridine, can significantly accelerate the reaction. Example 17 serves as a comparative example to the addition of 2,2′-bipyridine. To a 300 mL autoclave equipped with a liquid sampling loop and a high pressure addition funnel was added 0.132 g (0.5 mmol) of RhCl₃.3H₂O, 0.086 g (0.55 mmol) of 2,2′-bipyridine (Aldrich), 110.5 g (0.50 mol) of N-methylpyridinium iodide, and 45.0 g (0.75 mol) of acetic acid. To the addition funnel was added 64.0 g (2.0 mol) of methanol. The autoclave was flushed with nitrogen and was then heated to 190° C. under 17.2 barg (250 psig) of 5% hydrogen in carbon monoxide and the addition funnel heated to 150° C. Upon reaching 190° C., the gas feed was switched to 100% CO the methanol was added by pressurizing the addition funnel. Immediately upon completing the liquid addition, the pressure was adjusted to 51.7 barg (750 psig) using 100% CO. Samples were removed from the autoclave every 30 minutes for a period of 4 hrs (at which time the reaction was complete). The temperature and pressure were maintained using 100% CO as needed to maintain pressure. GC analysis of the liquid products is shown in Table IV below. Included in this chart is the number of moles of the acetyl components, methyl acetate, and acetic acid. The net acetyls produced is represented by the total of the acetic acid and methyl acetate present minus the 0.75 mol of acetic acid added at the start of the reaction. The rate of production in this reaction (determined from the slope of a graph of net moles of acetyl produced moles vs. time) was 0.513 mol/h representing a space time yield of 2.34 mol acetyl/L-h and a Rh turnover frequency of 1026 mol of acetyl/mol-Rh/h.

TABLE IV

| | GC Analysis | | | GC Analysis | | Net Acetyls |
|---|---|---|---|---|---|---|
| Time | (wt. %) | | | (moles) | | Produced |
| (hrs) | MeOH | MeOAc | AcOH | MeOAc | AcOH | (moles) |
| 0.5 | 8.7 | 15.6 | 13.7 | 0.48 | 0.52 | 0.25 |
| 1.0 | 1.4 | 16.8 | 21.3 | 0.54 | 0.84 | 0.63 |
| 1.5 | 0.3 | 10.7 | 32.3 | 0.36 | 1.32 | 0.93 |
| 2.0 | 0.1 | 6.3 | 39.9 | 0.22 | 1.67 | 1.14 |
| 2.5 | 0 | 2.7 | 45.6 | 0.09 | 1.95 | 1.29 |
| 3.0 | 0 | 0.6 | 51.2 | 0.02 | 2.23 | 1.50 |
| 3.5 | 0 | 0.3 | 62.0 | 0.01 | 2.89 | 2.15 |
| 4.0 | 0 | 0.2 | 59.3 | 0.01 | 2.72 | 1.97 |

### Examples 19-29

Example 18 was repeated except that various bidentate ligands were substituted for the 2,2′-bipyridyl ligand. The amount of bidentate ligand used was consistent on a molar basis in each reaction. The rates and turnover frequencies based on the graphs of the moles of acetyl produced vs. time for Examples 17-29 are summarized in Table V below.

TABLE V

| | | Rate | Turnover |
|---|---|---|---|
| | | (mol | Freq. |
| Example | | acetyl/ | (mol acetyl/ |
| No. | Ligand | L-h) | mol Rh/h) |
| 17 | none | 0.49 | 213 |
| 18 | 2,2′-bipyridine | 2.34 | 1026 |
| 19 | 2,3-Bis(2,6-di-i-propylphenylimino) | 1.73 | 758 |
| | butane | | |
| 20 | 4,7-diphenyl-1,10-phenanthroline | 0.95 | 415 |
| 21 | 1,10-phenanthroline | 0.61 | 268 |
| 22 | diphenyl-2-pyridylphosphine | 0.83 | 362 |
| 23 | 1,3-bis(diphenylphosphino)propane | 1.44 | 633 |
| 24 | 1,2-bis(diphenylphosphino)benzene | 1.03 | 452 |
| 25 | 1,4-bis(diphenylphosphino)butane | 0.99 | 434 |
| 26 | 1,6-bis(diphenylphosphino)hexane | 0.95 | 418 |
| 27 | N,N-bis(diphenylphosphino)amine | 0.94 | 413 |
| 28 | 1,5-bis(diphenylphosphino)pentane | 0.82 | 360 |
| 29 | bis(diphenylphosphino)methane | 0.72 | 317 |

As seen from the results in Table V, the examples that employed a bidentate ligand showed a higher reaction rate and catalyst turnover frequency compared to the example that did not employ a bidendate ligand.

The invention has been described in detail with particular reference to preferred embodiments thereof, but it will be understood that variations and modifications can be effected within the spirit and scope of the invention.

We claim:

1. A process for producing a carboxylic acid, a carboxylic acid ester, a carboxylic acid anhydride, or a mixture thereof, said process comprising contacting:
(i) a carbonylation feedstock compound selected from alkanols, dialkyl ethers, carboxylic acid esters, and mixtures thereof;
(ii) a Group VIII metal carbonylation catalyst;
(iii) an onium salt compound;
(iv) a bidentate ligand comprising two functional groups selected from tertiary amines and tertiary phosphines; and
(v) carbon monoxide,
in a reaction zone at conditions effective to produce a carbonylation product selected from a carboxylic acid, a carboxylic acid ester, a carboxylic acid anhydride, and a mixture thereof,
wherein a halide compound, other than the onium salt compound, exogenous or extraneous to the process is not added or supplied to the reaction zone.

2. A process according to claim 1, wherein the process is carried out at a pressure (total) of about 5 to 100 bar gauge (barg) and a temperature of about 50 to 300° C.

3. A process according to claim 2, wherein the Group VIII metal carbonylation catalyst is rhodium, iridium, or a compound thereof; and the onium salt compound is a quaternary ammonium halide or a quaternary phosphonium halide.

4. A process according to claim 1, wherein the process is carried out at a pressure (total) of about 5 to 100 bar gauge and a temperature of about 150 to 250° C.; the carbonylation product is acetic acid, methyl acetate, acetic anhydride, or a mixture thereof; the carbonylation feedstock compound is methanol, dimethyl ether, methyl acetate, or a mixture thereof; the Group VIII metal carbonylation catalyst is rhodium or a compound thereof; the onium salt compound is a 1,3-dialkylimidazolium iodide or an N-alkylpyridinium iodide; and the bidentate ligand is 2,2′-bipyridine, a diphosphine, a 2,2′-bipyridine, or a diimine.

5. A process for producing a carboxylic acid, a carboxylic acid ester, a carboxylic acid anhydride, or a mixture thereof, said process comprising:
(a) feeding to a reaction zone
(i) a carbonylation feedstock compound selected from alkanols, dialkyl ethers, carboxylic acid esters, and mixtures thereof,
(ii) a Group VIII metal carbonylation catalyst,
(iii) an onium salt compound,
(iv) a bidentate ligand selected from 2,2′-dipyridine, a 2,2′-dipyridine, a diimine, and a diphosphine, and
(v) optionally, an inert solvent, to provide a reaction zone liquid;
(b) feeding carbon monoxide to the reaction zone liquid under carbonylation conditions of pressure and temperature; and
(c) removing from the reaction zone a crude liquid product comprising a carbonylation product, the carbonylation feedstock compound, the Group VIII metal carbonylation catalyst, the onium salt compound, the bidentate ligand, and the optional inert solvent;
wherein a halide compound, other than the onium salt compound, exogenous or extraneous to the process is not added to the reaction zone.

6. A process according to claim 5, wherein the Group VIII metal carbonylation catalyst is rhodium, iridium, or a compound thereof; and the onium salt compound is a quaternary ammonium halide or a quaternary phosphonium halide.

7. A process according to claim 6, wherein the reaction zone liquid comprises about 10 to 80 weight percent of the carbonylation feedstock compound, about 10 to 80 weight percent of the carbonylation product, about 10 to 80 weight percent of the onium salt compound, and about 0 to 50 weight percent of the inert solvent.

8. A process according to claim 7, wherein the carbonylation product is acetic acid, methyl acetate, acetic anhydride, or a mixture thereof; the carbonylation feedstock compound is methanol, dimethyl ether, methyl acetate, or a mixture thereof;
the Group VIII metal carbonylation catalyst is rhodium or a compound thereof; and the onium salt compound is a 1,3-dialkylimidazolium iodide or an N-methylpyridinium salt.

9. A process according to claim 8, which further comprises:
(d) refining the crude liquid carbonylation product to recover (1) the carbonylation product, (2) a low-boiling fraction comprising the carbonylation feedstock compound, and (3) a high-boiling fraction comprising the Group VIII metal carbonylation catalyst, the onium salt compound, the bidentate ligand, and the optional inert solvent; and
(e) recycling the low-boiling and the high-boiling fractions to the reaction zone.

10. A process for producing a carboxylic acid, a carboxylic acid ester, a carboxylic acid anhydride, or a mixture thereof, said process comprising:
(a) feeding a carbonylation feedstock compound selected from alkanols, dialkyl ethers, carboxylic acid esters, and mixtures thereof and carbon monoxide to a reaction zone containing a solution comprising a Group VIII metal carbonylation catalyst, an onium salt compound, and a bidentate ligand comprising two functional groups selected from tertiary amines and tertiary phosphines to provide a reaction zone liquid maintained under carbonylation conditions of pressure and temperature; and
(b) removing from the reaction zone a crude gaseous product comprising a carbonylation product, the carbonylation feedstock compound, and carbon monoxide,
wherein a halide compound, other than the onium salt compound, exogenous or extraneous to the process is not added to the reaction zone.

11. A process according to claim 10, wherein the Group VIII metal carbonylation catalyst is rhodium, iridium, or a compound thereof; and the onium salt compound is a quaternary ammonium halide or a quaternary phosphonium halide.

12. A process according to claim 11, wherein the carbonylation product is acetic acid, methyl acetate, acetic anhydride, or a mixture thereof; the carbonylation feedstock compound is methanol, dimethyl ether, methyl acetate, or a mixture thereof;
the Group VIII metal carbonylation catalyst is rhodium or a compound thereof; the onium salt compound is a 1,3-dialkylimidazolium iodide or an N-methylpyridinium salt;
and the bidentate ligand is 2,2′-bipyridine, a diphosphine, a 2,2′-bipyridine, or a diimine.

13. A process according to claim 12, which further comprises:
(c) refining the crude gaseous carbonylation product to recover (1) the carbonylation product and (2) a low-boiling fraction comprising the carbonylation feedstock compound; and
(d) recycling the low-boiling fraction to the reaction zone.

14. A process for producing a carboxylic acid, a carboxylic acid ester, a carboxylic acid anhydride, or a mixture thereof, said process comprising:
(a) feeding a gaseous carbonylation feedstock compound selected from alkanols, dialkyl ethers, carboxylic acid esters, and mixtures thereof and carbon monoxide to a reaction zone containing a Group VIII metal carbonylation catalyst, an onium salt compound, and a bidentate ligand comprising two functional groups selected from tertiary amines and tertiary phosphines (1) deposited on a catalyst support material or (2) in the form of a polymeric material containing quaternary nitrogen groups; and
(b) removing from the reaction zone a crude gaseous product comprising a carbonylation product, the carbonylation feedstock compound, and carbon monoxide,
wherein a halide compound, other than the onium salt compound, exogenous or extraneous to the carbonylation process is not added to the reaction zone.

15. A process according to claim 14, wherein the carbonylation product is a carboxylic acid or a carboxylic acid anhydride; the carbonylation feedstock compound is an alkanol, a dialkyl ether, an alkyl carboxylic acid ester, or a mixture thereof; the Group VIII metal carbonylation catalyst is rhodium, iridium, or a compound thereof; the onium salt compound is a quaternary ammonium halide or a quaternary phosphonium halide;
and the bidentate ligand is 2,2′-bipyridine, a 2,2′-bipyridine, a diimine, or a diphosphine.

16. A process according to claim 15, which further comprises:
(c) refining the crude gaseous carbonylation product to recover (1) the carbonylation product and (2) a low-boiling fraction comprising the carbonylation feedstock compound; and
(d) recycling the low-boiling fraction to the reaction zone.
